(19) United States
(12) Patent Application Publication
(10) Pub. No.: US 2024/0081669 A1
(43) Pub. Date: Mar. 14, 2024

(54) DRIVE CIRCUIT FOR MEASURING BLOOD PRESSURE, AND BLOOD PRESSURE MEASUREMENT DEVICE

(71) Applicant: OMRON HEALTHCARE Co., Ltd., Kyoto (JP)

(72) Inventors: Shohei IWATA, Kyoto (JP); Takanori NISHIOKA, Kyoto (JP)

(21) Appl. No.: 18/511,542

(22) Filed: Nov. 16, 2023

Related U.S. Application Data
(63) Continuation of application No. PCT/JP2022/023545, filed on Jun. 10, 2022.

(30) Foreign Application Priority Data
Jun. 29, 2021 (JP) 2021-107230

(51) Int. Cl. A61B 5/0235

(52) U.S. Cl. CPC A61B 5/0235

(57) ABSTRACT

A drive circuit for measuring blood pressure that generates a first drive signal for driving a valve for opening and closing a flow path connected to a blood pressure measurement cuff and a second drive signal for driving a pump for supplying a fluid to the cuff. The first drive signal and the second drive signal are generated from a common power supply voltage supplied from a power source circuit. Envelopes thereof have a common shape changing at a same timing.

### CROSS-REFERENCE TO RELATED APPLICATIONS

This application is the U.S. national stage application filed pursuant to 35 U.S.C. 365(c) and 120 as a continuation of International Patent Application No. PCT/JP2022/023545, filed Jun. 10, 2022, which application claims priority to Japanese Patent Application No. 2021-107230, filed Jun. 29, 2021, which applications are incorporated herein by reference in their entireties.

### TECHNICAL FIELD

The present invention relates to a drive circuit for measuring blood pressure and a blood pressure measurement device used for blood pressure measurement.

### BACKGROUND ART

In recent years, blood pressure measurement devices used for measuring a blood pressure are being used as means to check health status at home, as well as in medical facilities. A blood pressure measurement device detects vibration of the artery wall to measure blood pressure by, for example, inflating and contracting a cuff wound around the upper arm or the wrist of a living body and detecting the pressure in the cuff by using a pressure sensor.

As such a blood pressure measurement device, there is known a technique that the blood pressure measurement device includes a sensing cuff for measuring a blood pressure and a plurality of cuffs including a pressing cuff that presses the sensing cuff against a living body. The blood pressure measurement device includes a pump and supplies a fluid, for example, air to the cuff by the pump. JP 2013-220288 A discloses a configuration in which, for example, a blood pressure measurement device includes an exhaust valve to exhaust air supplied to a cuff. For example, the pump is a piezoelectric pump that includes a piezoelectric element and a diaphragm coupled to the piezoelectric element, when an AC voltage is applied, the piezoelectric element vibrates, and the diaphragm vibrates by the vibration of the piezoelectric element to pump out a fluid. For example, the valve is a capacitive type, and is a normally-open type exhaust valve in which a valve body opens a flow path when not energized.

Such a blood pressure measurement device includes a pump drive circuit for driving the pump and a valve drive circuit for driving the valve. When an instruction to start blood pressure measurement is input, a processor of the blood pressure measurement device outputs a control signal to the valve drive circuit. The valve drive circuit closes the valve based on the control signal. Thereafter, the processor outputs the control signal to the pump drive circuit. The pump drive circuit drives the pump based on the control signal and performs control to send air to a cuff. The pump drive circuit inflates the cuff with the air supplied by the pump to gradually pressurize the cuff. The blood pressure measurement device calculates a blood pressure value from a pressure of the cuff detected by a pressure sensor. After calculating the blood pressure value, the processor outputs a signal for stopping the pump to the pump drive circuit, and the pump drive circuit stops the pump. Further, the processor outputs a control signal for opening the valve to the valve drive circuit. The valve drive circuit opens the valve, and thus the air inside the cuff is exhausted. In this way, the blood pressure measurement device controls the valve and the pump by the valve drive circuit and the pump drive circuit according to the control signals from the processor, and measures the pressure of the cuff required for blood pressure measurement.

### CITATION LIST

### Patent Literature

Patent Literature 1: JP 2013-220288 A

### SUMMARY OF INVENTION

### Technical Problem

Recently, as a wearable device attached to a wrist, there is a demand for a reduction in the size of the blood pressure measurement device. However, in the above-described conventional blood pressure measurement device, since the pump and the valve have different drive voltages, the drive circuits for the pump and the valve are constituted by different circuit blocks. During blood pressure measurement, the processor outputs individual control signals to the respective drive circuits to control the pump and valve.

When the pump drive circuit and the valve drive circuit are configured as the different circuit blocks as described above, the circuit areas of the drive circuits increase and the number of components also increases. This is a factor that hinders reduction in size of the blood pressure measurement device.

Therefore, an object of the present invention is to provide a drive circuit for measuring blood pressure and a blood pressure measurement device that can be reduced in size.

### Solution to Problem

According to one aspect, there is provided a drive circuit for measuring blood pressure that generates a first drive signal for driving a valve for opening and closing a flow path connected to a blood pressure measurement cuff and a second drive signal for driving a pump for supplying a fluid to the cuff. The first drive signal and the second drive signal are generated from a common power supply voltage supplied from a power source circuit. A waveform of the first drive signal and an envelope of a peak voltage of the second drive signal have a common shape changing at a same timing.

Here, the fluid includes a liquid and air. The cuff includes a bag-like structure that is wound around, for example, an upper arm and a wrist of a living body when a blood pressure is measured and inflates by supply of a fluid. When the fluid is air, the bag-like structure is an air bag inflated by, for example, air.

According to this aspect, the valve connected to the cuff and the pump that supplies the fluid to the cuff can be driven with the first drive signal and the second drive signal generated by the common power supply voltage supplied from the power source circuit. The waveform of the first drive signal and the envelope of the second drive signal have the common shape changing at the same timing. Therefore, without providing drive circuits to the respective pump and valve, the pump and the valve can be driven by one drive circuit for measuring blood pressure. Therefore, the use of the drive circuit for the blood pressure measurement device allows reducing the size of the blood pressure measurement device and reducing the number of components.

There is provided a drive circuit for measuring blood pressure according to the one aspect that includes a control circuit and a transformer circuit. The control circuit outputs the second drive signal. The transformer circuit transforms the power supply voltage into a voltage value corresponding to the first drive signal and the second drive signal and outputs the transformed voltage to the control circuit and the valve.

According to this aspect, the pump and the valve can be driven by the voltage transformed by the transformer circuit. Therefore, the drive circuit allows integrating the control circuit and the transformer circuit. That is, since the control circuit and the transformer circuit can be provided in one circuit block, the size of the driver circuit can be reduced and the number of components can be reduced. Therefore, by using the drive circuit in the blood pressure measurement device, it is possible to reduce the size of the blood pressure measurement device.

There is provided a drive circuit for measuring blood pressure according to the one aspect in which the transformer circuit is a booster circuit that boosts the power supply voltage.

According to this aspect, even when a voltage output from the power source circuit is a voltage lower than the drive signal of the pump and the valve, the drive circuit for measuring blood pressure can boost the voltage to a voltage for driving the pump and the valve.

There is provided a drive circuit for measuring blood pressure according to the one aspect in which the transformer circuit gradually increases the voltage value output to the valve and the control circuit.

According to this aspect, by gradually increasing the voltage for driving the cuff, the drive circuit for measuring blood pressure can pressurize the cuff by supplying a fluid to an inside of the cuff at a constant speed that is preferable when blood pressure measurement is performed.

There is provided a drive circuit for measuring blood pressure according to the one aspect in which a voltage value at which the valve is driven is higher than a voltage at a start of driving the pump. The transformer circuit transforms the power supply voltage into the voltage value at which the valve is driven and subsequently transforms the voltage value to a voltage value at which driving of the valve is maintained and the pump is driven.

According to this aspect, even when the voltage for driving the valve is higher than the voltage at the start of driving the pump, the drive circuit for measuring blood pressure can drive the valve and the pump using the same transformer circuit and the same control circuit. Thus, the drive circuit for measuring blood pressure can be reduced in size.

There is provided a drive circuit for measuring blood pressure according to the one aspect in which the control circuit outputs a PWM signal to the pump and the valve as the first drive signal and the second drive signal. The PWM signal has an effective voltage equal to or more than a voltage required for the valve and the pump to operate.

According to this aspect, the control circuit can set, as the first drive signal and the second drive signal, the PWM signal having the effective voltage equal to or more than the voltage required to operate the pump that supplies a fluid to the cuff and the valve connected to the cuff. Therefore, since the control circuit can be provided in one circuit block, the size of the driver circuit that drives the pump and the valve can be reduced and the number of components can be reduced. Therefore, the use of the drive circuit for measuring blood pressure for the blood pressure measurement device allows reducing the size of the blood pressure measurement device.

There is provided a drive circuit for measuring blood pressure according to the one aspect in which the control circuit gradually increases a voltage value of the effective voltage output to the valve and the pump.

According to this aspect, by gradually increasing the voltage for driving the cuff, the drive circuit for measuring blood pressure can pressurize the cuff by supplying a fluid to an inside of the cuff at a constant speed that is preferable when blood pressure measurement is performed.

There is provided a drive circuit for measuring blood pressure according to the one aspect in which a voltage value at which the valve is driven is higher than a voltage at a start of driving the pump. The control circuit sets the effective voltage to the voltage value at which the valve is driven and subsequently sets the voltage value to a voltage value at which driving of the valve is maintained and the pump is driven.

According to this aspect, even when the voltage for driving the valve is higher than the voltage at the start of driving the pump, the drive circuit for measuring blood pressure can drive the valve and the pump using the same control circuit. Thus, the drive circuit for measuring blood pressure can be reduced in size.

According to an aspect, there is provided a blood pressure measurement device that includes a cuff, a pump, a valve, a power source circuit, the drive circuit for measuring blood pressure, and a processor. A fluid is supplied to the cuff. The pump supplies the fluid to the cuff. The valve opens and closes a flow path connected to the cuff. The drive circuit for measuring blood pressure is according to the one aspect. The processor outputs a control signal of a voltage to the drive circuit for measuring blood pressure.

According to this aspect, the pump that supplies the fluid to the cuff and the valve connected to the cuff can be driven with the first drive signal and the second drive signal generated by the common power supply voltage supplied from the power source circuit. Therefore, without providing drive circuits to the respective pump and valve, the pump and the valve can be driven by one drive circuit for measuring blood pressure. Therefore, since the drive circuit for measuring blood pressure can be provided in one circuit block, the size of the driver circuit for measuring blood pressure be reduced and the number of components can be reduced. Thus, the blood pressure measurement device can be reduced in size.

### Advantageous Effects of Invention

The present invention can provide the drive circuit for measuring blood pressure and the blood pressure measurement device that allow micrifying a circuit area and reducing the number of components.

### BRIEF DESCRIPTION OF DRAWINGS

FIG. 1 is a perspective view illustrating a configuration of a blood pressure measurement device according to a first embodiment of the present invention.

FIG. 2 is a block diagram schematically illustrating a configuration of a device body of the blood pressure measurement device.

FIG. 3 is a block diagram illustrating a configuration of a main part of the blood pressure measurement device.

FIG. 4 is an explanatory diagram illustrating an example of control during blood pressure measurement using the blood pressure measurement device.

FIG. 5 is a flowchart depicting an example of usage of the blood pressure measurement device.

FIG. 6 is a perspective view illustrating a state in which the blood pressure measurement device is attached to a wrist.

FIG. 7 is an explanatory diagram illustrating an example of control during blood pressure measurement using a blood pressure measurement device according to a second embodiment of the present invention.

FIG. 8 is a block diagram illustrating a configuration of a main part of a blood pressure measurement device according to a third embodiment of the present invention.

### DESCRIPTION OF EMBODIMENTS

### First Embodiment

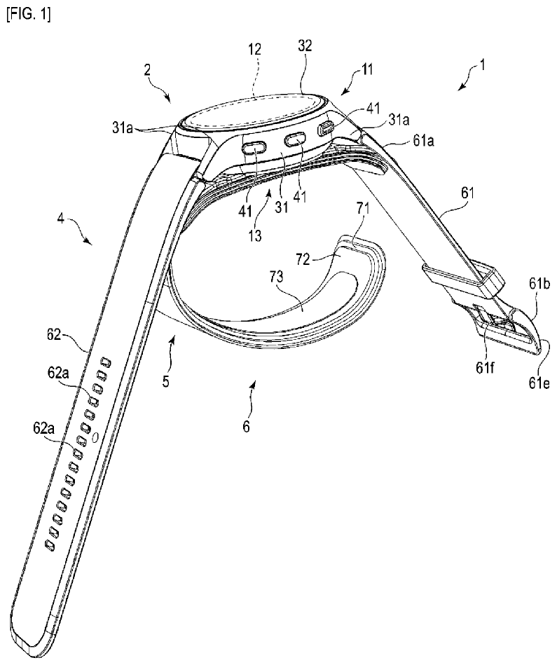

An example of a blood pressure measurement device 1 according to the first embodiment of the present invention will be described below with reference to FIG. 1 to FIG. 6.

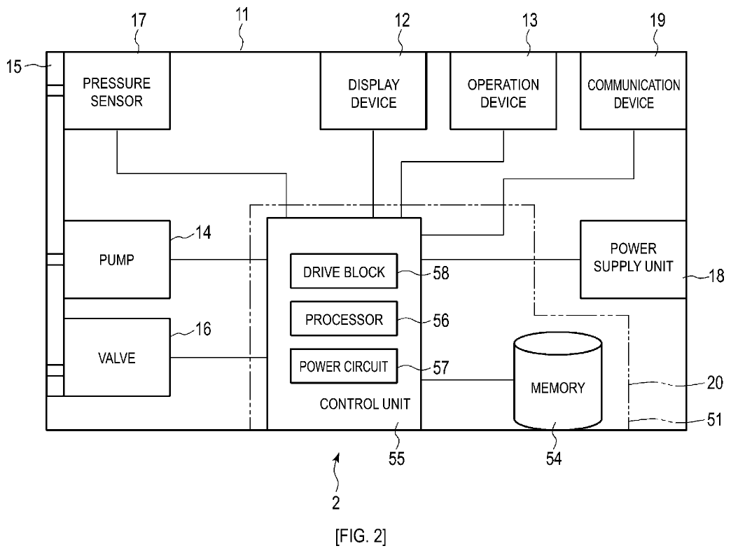

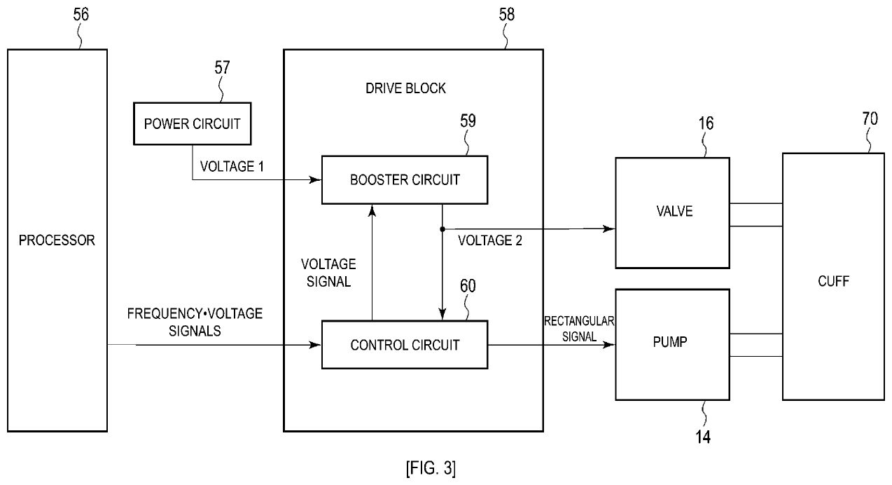

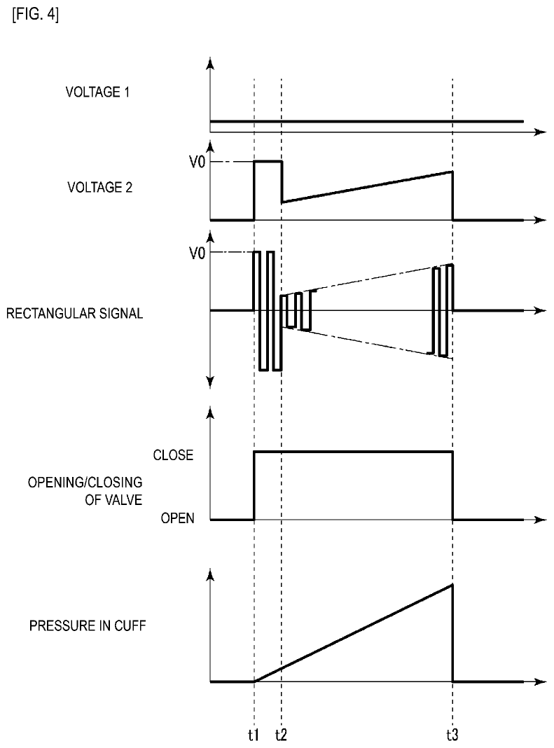

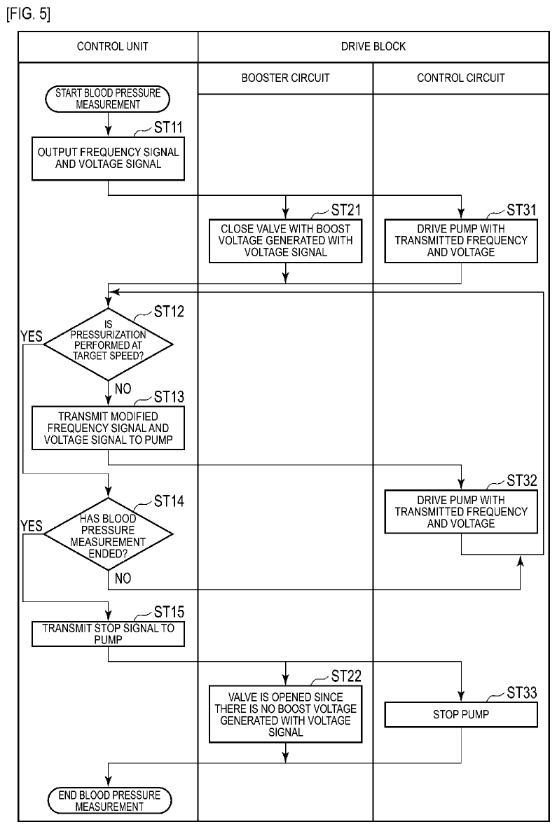

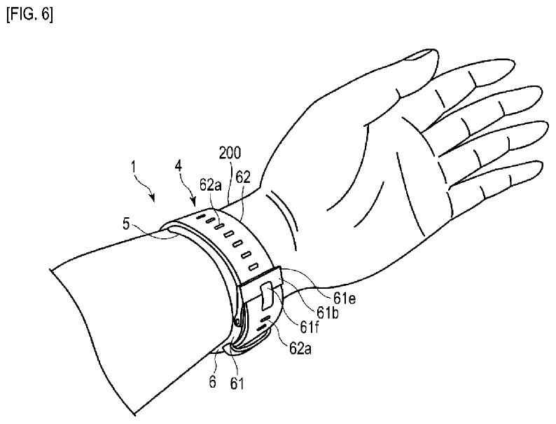

FIG. 1 is a perspective view illustrating a configuration of the blood pressure measurement device 1 according to a first embodiment of the present invention. FIG. 2 is a block diagram schematically illustrating a configuration of a device body 2 of the blood pressure measurement device 1. FIG. 3 is a block diagram schematically illustrating a configuration of a processor 56, a power source circuit 57, a drive block 58, a pump 14, and a valve 16 of the blood pressure measurement device 1. FIG. 4 is an explanatory diagram illustrating an example of control during blood pressure measurement using the blood pressure measurement device 1. FIG. 5 is a flowchart depicting an example of blood pressure measurement of the blood pressure measurement device 1. FIG. 6 is a perspective view illustrating a state in which the blood pressure measurement device 1 is attached to a wrist 200.

The blood pressure measurement device 1 is an electronic blood pressure measurement device attached to a living body. The blood pressure measurement device 1 is an electronic blood pressure measurement device that is attached to the living body 200, such as a wrist, and has an aspect of measuring a blood pressure from arteries of the living body 200.

As illustrated in FIG. 1 to FIG. 3, the blood pressure measurement device 1 includes the device body 2, a fixture 4, such as a belt, a curler 5 disposed between the fixture 4 and the living body 200, and a cuff structure 6 including a cuff 70. In the present embodiment, an example in which the blood pressure measurement device 1 is attached to the wrist 200 as the living body 200 will be described, but the living body 200 may be an upper arm or the like.

As illustrated in FIG. 1 and FIG. 2, the device body 2 includes a case 11, a display device 12, an operation device 13, the pump 14, a flow path unit 15, the valve 16, a pressure sensor 17, a power supply unit 18, a communication device 19, and a control board 20.

The case 11 houses, for example, the display device 12, the operation device 13, the pump 14, the flow path unit 15, the valve 16, the pressure sensor 17, the power supply unit 18, the communication device 19, and the control board 20. Additionally, the case 11 exposes a portion of the display device 12 or a portion thereof is made of a transparent material such that a portion of the display device 12 can be visually recognized from the outside.

The case 11 includes, for example, an outer case 31 and a windshield 32 that covers an opening of the outer case 31 on a side opposite to the wrist 200 side (outer side). In addition, the case 11 may include a base portion provided inside the outer case 31, a back cover that covers the wrist 200 side of the outer case 31, a sealing member that makes the case 11 liquid-tight, and the like.

The outer case 31 is formed in a cylindrical shape. The outer case 31 includes, for example, pairs of lugs 31a provided at respective symmetrical positions in a circumferential direction of an outer circumferential surface and respective spring rods provided between the two pairs of lugs 31a. The windshield 32 is, for example, a circular glass plate.

The display device 12 is electrically connected to the control board 20. The display device 12 is, for example, a Liquid Crystal Display (LCD) or an Organic Electro Luminescence Display (OELD). The display device 12 displays various types of information including date and time and measurement results of, for example, blood pressure values, such as a systolic blood pressure and a diastolic blood pressure, and a heart rate in response to a control signal from the control board 20.

A user inputs an instruction with the operation device 13. For example, the operation device 13 includes a plurality of buttons 41. The operation device 13 includes a sensor that detects an operation of the button 41, a pressure-sensitive type touch panel, a capacitive touch panel, or the like provided on, for example, the case 11 and the display device 12, a microphone for receiving an instruction by sound, and the like. When operated by the user, the operation device 13 converts the instruction into an electrical signal and outputs the electrical signal to the control board 20.

The pump 14 is, for example, a piezoelectric pump. The pump 14 compresses a fluid and supplies the compressed fluid to the cuff 70 through the flow path unit 15. The pump 14 is electrically connected to the control board and driven based on a control signal (second drive signal) provided from the control board 20.

As a specific example, the pump 14 is a pump that includes a piezoelectric element and a diaphragm coupled to the piezoelectric element, when an AC voltage as a drive signal is applied to the piezoelectric element, the diaphragm vibrates together with the piezoelectric element, and the vibration of the diaphragm pumps out the fluid. The drive signal is, for example, a rectangular signal. Additionally, any gas or any liquid can be employed as the fluid. In the present embodiment, the fluid is air. Since the piezoelectric pump is small and thin, the use of the piezoelectric pump as the pump 14 allows reducing the size of the blood pressure measurement device 1.

The flow path unit 15 connects the pump 14, the valve 16, and the pressure sensor 17 to the cuff 70. The flow path unit 15 is any of, for example, a tube, a pipe, a tank, and a hollow portion and a groove formed in the case 11, or a combination thereof. Note that the fluid circuit configuration of the flow path unit 15 and the cuff 70 is appropriately designed according to various factors, such as the way of flowing a fluid, the number and the configuration of the cuffs 70, the supply order of the plurality of cuffs 70, the exhaust method of the plurality of cuffs 70, and the blood pressure measurement method 5.

The valve 16 is electrically connected to the control board 20, and is opened and closed based on a drive voltage (first drive signal) supplied from the control board 20. The valve 16 opens and closes the flow path to the cuff 70. The valve 16 is connected to the atmosphere by the flow path unit 15 and is switched to an open state to connect the cuff 70 to the atmosphere and exhaust the air in the cuff 70.

The valve 16 is, for example, a quick exhaust valve to set an opening degree of the valve 16 or an opening area of the flow path unit 15 such that a fluid resistance becomes low as much as possible and that allows quick exhaust. The valve 16 is switched to a closed state when air is supplied to the cuff 70 during blood pressure measurement. In addition, when the air in the cuff 70 is exhausted, the valve 16 is controlled by the control board 20 so as to be switched from the closed state to the open state. Further, the valve 16 may be formed such that the opening degree is adjustable.

As a specific example, the valve 16 is a normally-open type that is normally open and closed by application of a predetermined voltage. As the valve 16, for example, an electrostatic drive type using a Micro Electro Mechanical System (MEMS) technology is used. Some electrostatic drive type valve have hysteresis characteristics in the drive voltage. That is, for example, when the drive voltage is increased to once switch the valve in the normally open state from the open state to the closed state, the closed state is maintained by electrostatic force until a certain limit value is reached even when the drive voltage is reduced after that.

The pressure sensor 17 detects the pressure of the cuff 70, at least a pressure of a sensing cuff 73 described later among the plurality of cuffs 70 of the cuff structure 6 in the present embodiment. As a specific example, the pressure sensor 17 is fluidly connected to the sensing cuff 73 via the flow path unit 15, and detects the pressure inside the sensing cuff 73. The pressure sensor 17 is electrically connected to the control board 20. The pressure sensor 17 outputs an electrical signal corresponding to the detected pressure to the control board 20.

The power supply unit 18 is a power source. The power supply unit 18 is, for example, a secondary battery such as a lithium ion battery. The power supply unit 18 is electrically connected to the control board 20. As a specific example, the power supply unit 18 supplies power to the control board 20. The power supply unit 18 supplies power for driving to the respective configurations of the control board 20 and the display device 12, the operation device 13, the pump 14, the valve 16, the pressure sensor 17, and the communication device 19 via the control board 20.

The communication device 19 can transmit and receive information to and from an external device wirelessly or by wire. The communication device 19 transmits information, such as information controlled by the control board and measured blood pressure values and pulse, to an external device, or receives, for example, a recording medium for software update from an external device and transmits this to the control unit.

In the present embodiment, the external device is, for example, an external terminal, such as a smartphone, a tablet terminal, a personal computer, and a smart watch.

In the present embodiment, the communication device 19 and the external device may be directly connected, or may be connected over a network. The communication device 19 and the external device may be connected via a mobile communication network, such as 4G and 5G, and a wireless communication line, such as Wimax and Wi-Fi (registered trademark). Further, the communication device 19 and the external device may be connected by wireless communication means, such as Bluetooth (registered trademark), Near Field Communication (NFC), and infrared communication. Furthermore, the communication device 19 and the external device may be connected over a wired communication line, such as a Universal Serial Bus (USB) and a Local Area Network (LAN) connection with a cable. Thus, the communication device 19 may include a plurality of communication means, such as a wireless antenna and a micro-USB connector.

As illustrated in FIG. 2, the control board 20 includes, for example, a substrate 51, a storage unit 54, and a control unit 55. The control board 20 is constituted by mounting the storage unit 54 and the control unit 55 on the substrate 51.

The substrate 51 is housed in the case 11.

The storage unit 54 is a memory mounted on the substrate 51. The storage unit 54 includes, for example, a Random Access Memory (RAM) and a Read Only Memory (ROM). The storage unit 54 stores various types of data. For example, the storage unit 54 pre-stores recording medium data for controlling the overall blood pressure measurement device 1 and the fluid circuit including the pump 14 and the valve 16, setting data for setting various functions of the blood pressure measurement device 1, calculation data for calculating a blood pressure value and a pulse from the pressure measured by the pressure sensor 17, and the like to be changeable. The storage unit 54 stores information, such as a measured blood pressure value, a measured value of, for example, a pulse, and a pressure value measured by the pressure sensor 17.

The control unit 55 includes a single or a plurality of the processors 56, the power source circuit 57, and the drive block 58 mounted on the substrate 51. For example, the processor 56 is a Central Processing Unit (CPU). The control unit 55 controls the operation of the overall blood pressure measurement device 1 and the operations of the pump 14 and the valve 16 based on the recording mediums stored in the storage unit 54 and various circuits, such as the power source circuit 57 and the drive block 58, to perform a predetermined operation (function). In addition, in accordance with the read recording medium, the control unit 55 performs, for example, predetermined operation, analysis, or process in the control unit 55. The control unit 55 configures a portion or all of the respective functions performed by the control unit 55 in hardware by, for example, one or a plurality of integrated circuits.

As illustrated in FIG. 2, the control unit 55 is electrically connected to and supplies power to the display device 12, the operation device 13, the pump 14, the valve 16, and the pressure sensor 17. Additionally, the control unit 55 controls the operations of the display device 12, the pump 14, and the valve 16, based on electrical signals output by the operation device 13 and the pressure sensor 17. The control unit 55 controls the pump 14 and the valve 16 to supply air to the cuff 70, and calculates a blood pressure by an oscillometric method based on the pressure of the sensing cuff 73 detected by the pressure sensor 17.

For example, the processor 56 includes a main CPU that controls the operation of the overall blood pressure measurement device 1 and a sub-CPU that controls the operation of the fluid circuit. Note that, for example, the control unit 55 may be configured to perform all the controls of the blood pressure measurement device 1 in one CPU. For example, the processor 56 obtains measurement results, such as blood pressure values, for example, a systolic blood pressure and a diastolic blood pressure, and a heart rate, from the electrical signals output by the pressure sensor 17, and outputs image signals corresponding to the measurement results to the display device 12.

In addition, for example, when an instruction for measuring a blood pressure is input from the operation device 13, the processor 56 outputs instruction values, such as a frequency signal and a voltage signal for driving the pump 14 and the valve 16, to the drive block 58. The processor 56 controls driving and stopping of the pump 14 and opening and closing of the valve 16 based on electrical signals output by the pressure sensor 17. The processor 56 controls the pump 14 and the valve 16 to supply compressed air to the cuff 70 and selectively depressurize the cuff 70.

In the present embodiment, the frequency signal is a signal for designating a frequency of a rectangular wave for driving the pump 14. Further, in the present embodiment, the voltage signal is a signal for designating a voltage value for driving the pump 14 and the valve 16. The voltage signal is a signal common to the pump 14 and the valve 16, and is information on a voltage value output to the pump 14 and the valve 16.

In the present embodiment, the drive voltage for switching the valve 16 from the open state to the closed state is set higher than the voltage of the drive signal output to the pump 14 at the start of blood pressure measurement, and the drive voltage for switching from the closed state to the open state is set 0 V or lower than the voltage of the drive signal output to the pump 14 during blood pressure measurement.

The power source circuit 57 supplies the power supplied from the power supply unit 18 to the drive block 58. The power source circuit 57 may be configured to further supply power for driving to the display device 12, the operation device 13, the pressure sensor 17, the communication device 19, and the processor 56. For example, in addition to the power source circuit 57 that supplies the power supplied from the power supply unit 18 to the drive block 58, the control unit 55 may include a power source circuit that supplies the power supplied from the power supply unit 18 to the display device 12, the operation device 13, the pressure sensor 17, the communication device 19, and the processor 56.

The drive block 58 includes a transformer circuit 59 and a control circuit 60. The drive block 58 configures the transformer circuit 59 and the control circuit 60 in one circuit block. The drive block 58 is a drive circuit that drives the pump 14 and the valve 16 by the transformer circuit 59 and the control circuit 60. The drive block 58 is a drive circuit for measuring blood pressure.

The transformer circuit 59 transforms a power supply voltage supplied from the power source circuit 57. For example, the transformer circuit 59 is a booster circuit that boosts the power supply voltage supplied from the power source circuit 57. In the present embodiment, the transformer circuit 59 will be hereinafter described as the booster circuit 59.

As illustrated in FIG. 3, the booster circuit 59 is connected to the power source circuit 57, the control circuit 60, and the valve 16. An input of the booster circuit 59 is connected to the power source circuit 57 and the control circuit 60. An output of the booster circuit 59 is connected to the control circuit 60 and the valve 16.

The booster circuit 59 boosts the voltage input from the power source circuit 57 in accordance with the voltage signal output from the processor 56, and outputs it to the valve 16 and the control circuit 60. The voltage signal output from the processor 56 is input to the booster circuit 59 directly from the processor 56 or indirectly via the control circuit 60. The present embodiment will be described using an example in which the voltage signal output from the processor 56 is input to the booster circuit 59 via the control circuit 60.

The control circuit 60 is connected to the processor 56, the booster circuit 59, and the pump 14. An input of the control circuit 60 is connected to the processor 56 and the booster circuit 59. An output of the control circuit 60 is connected to the booster circuit 59 and the pump 14. When the frequency signal and the voltage signal output from the processor 56 are input, the control circuit 60 outputs the voltage signal to the booster circuit 59. The control circuit 60 also outputs the voltage boosted by the booster circuit 59 to the pump 14 as the rectangular signal based on the frequency signal.

As illustrated in FIG. 1 and FIG. 6, the fixture 4 is a so-called belt and includes a first belt 61 provided at the first pair of lugs 31a and the spring rod, and a second belt 62 provided at the second pair of lugs 31a and the spring rod. When the blood pressure measurement device 1 is attached to the wrist 200, the fixture 4 is wound around the wrist 200 via the curler 5.

The first belt 61 is referred to as a so-called a parent and is configured like a band capable of being joined to the second belt 62. The first belt 61 includes a belt portion 61a and a buckle 61b. The belt portion 61a is configured like a band. The belt portion 61a is formed of an elastically deformable resin material. In addition, the belt portion 61a is flexible and includes a sheet-like insert member inside the belt portion 61a for suppressing stretching in the longitudinal direction of the belt portion 61a.

The buckle 61b includes a frame body 61e in a rectangular frame shape and a prong 61f rotatably attached to the frame body 61e. One side of the frame body 61e to which the prong 61f is attached is mounted rotatably to the belt portion 61a. The first belt 61 is attached between the pair of lugs 31a via the spring rod and rotatably held to the outer case 31.

The second belt 62 is referred to as a so-called blade tip, and is configured in a band-like shape having a width at which the second belt 62 can be inserted into the frame body 61e. The second belt 62 is formed of an elastically deformable resin material. In addition, the second belt 62 is flexible and includes a sheet-like insert member inside the second belt 62 for suppressing stretching in the longitudinal direction of the second belt 62.

In addition, the second belt 62 includes a plurality of small holes 62a into which the prong 61f is inserted. The second belt 62 is attached between the pair of lugs 31a via the spring rod and rotatably held to the outer case 31.

When the first belt 61 and the second belt 62 are coupled, the fixture 4 comes to have an annular shape following along the circumferential direction of the wrist 200 along with the device body 2. The fixture 4 presses the curler against the wrist 200 to elastically deform the curler 5 such that the curler 5 follows along the circumferential direction of the wrist 200 of a wearer of the blood pressure measurement device 1.

The curler 5 is configured in a band-like shape that curves following along the circumferential direction of the wrist 200. The curler 5 is formed with a first end and a second end spaced apart from each other. For example, a first end-side outer surface of the curler 5 is fixed to the device body 2. The curler 5 is disposed at a position where the first end and the second end protrude to one lateral side of the wrist 200. Accordingly, the curler 5 is disposed with the first end and the second end to one side of the wrist 200 when the blood pressure measurement device 1 is attached to the wrist 200. Furthermore, the first end and the second end of the curler 5 are located adjacent to each other at a predetermined distance from each other. The curler is formed of a resin material, for example.

As a specific example, the curler 5 is configured in a belt shape that curves to follow the circumferential direction of the wrist. As a specific example, the shorter side of the curler 5 from the device body 2 to the first end is disposed on the hand back-side of the wrist, and the longer side from the device body 2 to the second end extends from the hand back-side of the wrist, passing through one side, to the hand palm-side of the wrist 200.

The cuff structure 6 includes a plurality of the cuffs 70. The cuff structure 6 is wrapped around a wrist or the like of a living body to measure the blood pressure. The cuff 70 is a blood pressure measurement cuff. The cuff 70 includes a single or multi-layered bag-like structure to which a fluid is supplied. A fluid is supplied to the bag-like structure. In the present embodiment, since the fluid is air, the bag-like structure is an air bag. The bag-like structure is formed, for example, by stacking and welding a pair of sheet members.

For example, the cuff structure 6 includes a pressing cuff 71 as the cuff 70, a back plate 72, and the sensing cuff 73 as the cuff 70. The cuff structure 6 may include a tensile cuff as another cuff 70. The pressing cuff 71 is fluidly connected to the pump 14. The pressing cuff 71 is inflated by the air from the pump 14. The pressing cuff 71 is inflated to press the sensing cuff 73 against the living body. The pressing cuff 71 is formed by, for example, stacking a plurality of fluidly connected air bags in the pressing direction of the sensing cuff 73.

The back plate 72 is formed in a plate shape using a resin material. The back plate 72 has shape followability.

Here, the shape followability refers to a function of the back plate 72 by which the back plate 72 can be deformed in such a manner as to follow the shape of a contacted portion of the wrist 200 to be disposed, and the contacted portion of the wrist 200 refers to a region of the wrist 200 that is faced by the back plate 72. The contact used herein includes both direct contact of the back plate 72 to the wrist 200 and indirect contact to the wrist 200 via the sensing cuff 73. The back plate 72 is formed such that the length of the back plate 72 is sufficient to cover the palm side of the wrist 200. The back plate 72 presses the sensing cuff 73 by inflation of the pressing cuff 71 in a state where the back plate 72 extends along the shape of the wrist 200.

The sensing cuff 73 is supplied with air by the pump 14. The sensing cuff 73 is disposed in a region where the arteries of the wrist (living body) 200 are present when the blood pressure measurement device 1 is attached to the living body. The sensing cuff 73, which is used for detecting a pressure for calculating a blood pressure in blood pressure measurement, is supplied with air and is pressed by the inflated pressing cuff 71 to press the region where the artery of the wrist 200 is present. The sensing cuff 73 is formed by, for example, one air bag.

Next, an example of a relationship between the voltage, the rectangular signal, the opening/closing of the valve, and the pressure of the cuff 70 from a blood pressure measurement start time t1 until a blood pressure measurement end time t3 using the blood pressure measurement device 1 will be described below with reference to FIG. 4. In the blood pressure measurement in this example, an example in which correction of the rectangular signal by the pressure of the cuff 70 is not included will be described.

In FIG. 4, a voltage 1 is a voltage output from the power source circuit 57 to the booster circuit 59. A voltage 2 is a voltage output from the booster circuit 59 to the control circuit 60 and the valve 16. The rectangular signal is a signal for driving the pump 14 output from the control circuit 60 to the pump 14.

First, when an instruction to start blood pressure measurement is input by the operation device 13 or the like, the processor 56 outputs the frequency signal and the voltage signal as the drive signals of the pump 14 to the control circuit 60, and the control circuit 60 outputs the input voltage signal to the booster circuit 59. At this time, the processor 56 outputs the voltage signal having a voltage value at which the valve 16 is closed to the control circuit 60 as a signal at the start of blood pressure measurement.

The booster circuit 59 boosts the voltage 1 input from the power source circuit 57 to the voltage 2 for driving the valve 16 in the closing direction based on the voltage signal instructed from the control circuit 60, and outputs the boosted voltage to the control circuit 60 and the valve 16. The control circuit 60 generates the rectangular signal based on the voltage 2 and the frequency signal, and outputs the rectangular signal to the pump 14. Here, an electric signal output from the processor 56 is maintained at the voltage value at which the valve 16 is closed from the blood pressure measurement start time t1 until a predetermined time elapse time t2 at which the valve 16 is surely closed. This causes the valve 16 to close by the voltage 2 from t1 until t2. Then, the diaphragm of the pump 14 vibrates according to the voltage value and the frequency of the rectangular signal, thereby increasing the pressure in the cuff 70.

Here, a drive voltage VO for switching the valve 16 from the open state to the closed state is set higher than a drive voltage for switching the valve 16 from the closed state to the open state. The drive voltage for switching the valve 16 from the closed state to the open state is set to be lower than an amplitude value of the drive voltage of the pump 14. Therefore, by increasing the voltage 2 to VO at the start of blood pressure measurement to switch the valve 16 from the open state to the closed state, and after that by lowering the voltage 2 to a voltage required for driving the pump 14, driving of the pump 14 can be controlled while the valve 16 is maintained in the closed state. A time t2-t1 required for switching the valve 16 from the open state to the closed state is several ms to several tens of ms and is extremely short compared to a drive time t3-t1 of the pump 14. Therefore, an influence of the driving of the pump 14 by the drive voltage VO on the pressure control inside the cuff is negligibly small.

After the predetermined time elapse time t2 from the blood pressure measurement start time t1, the processor 56 outputs a voltage signal that is a voltage value for driving the pump 14 and corresponding to a voltage value at which the closed valve 16 does not open to the control circuit 60. At this time, the processor 56 outputs the voltage signal such that the voltage value for driving the pump 14 gradually increases. As a result, the closed state of the valve 16 is maintained from the predetermined time elapse time t2 until the blood pressure measurement end time t3, and the voltage 2 output from the booster circuit 59 gradually increases. The control circuit 60 generates the rectangular signal based on the input voltage 2. Therefore, the amplitude value of the rectangular signal input to the pump 14 gradually increases. Therefore, an amount of air supplied to the cuff 70 by the pump 14 gradually increases, and the pressure in the cuff 70 gradually increases.

When the blood pressure measurement ends, the processor 56 outputs a stop signal to the control circuit 60. For example, the stop signal is the frequency signal and the voltage signal, such as a frequency of 0 Hz and a voltage of 0 V. When the stop signal is input, the control circuit 60 stops generating the rectangular signal and outputs the stop signal to the booster circuit 59. When the stop signal is input, the booster circuit 59 stops boosting the voltage 1 supplied from the power source circuit 57 and stops outputting the voltage 2 to the valve 16 and the control circuit 60. Thus, the voltage input to the valve 16 becomes 0 V, and the valve 16 is not energized after the blood pressure measurement end time t3. Therefore, the valve 16 that has been closed opens, and thus the air in the cuff 70 is exhausted and the pressure in the cuff 70 becomes an atmospheric pressure.

Next, an example of blood pressure measurement using the blood pressure measurement device 1 will be described with reference to the flowchart depicted in FIG. 5.

First, for blood pressure measurement, as illustrated in FIG. 6, the user attaches the blood pressure measurement device 1 on the wrist 200 and turns on a power source of the blood pressure measurement device 1. Then, when the user operates the operation device 13 to input an instruction to start blood pressure measurement, the processor 56 outputs the frequency signal and the voltage signal (Step ST11).

The booster circuit 59 boosts the power supply voltage input from the power source circuit 57 by the voltage signal output by the processor 56 and input via the control circuit 60. Then, the booster circuit 59 outputs the generated boost voltage to the valve 16 to drive the valve 16 and close the valve 16 (Step ST21).

The control circuit 60 generates the rectangular signal from the frequency signal output from the processor 56 and the boost voltage generated by the booster circuit 59. Then, the control circuit 60 outputs the generated rectangular signal to the pump 14 and drives the pump 14 based on the rectangular signal (Step ST31). To supply air to the cuff 70 at a constant speed, the voltage signal output by the processor 56 is controlled such that the voltage boosted by the booster circuit 59 gradually increases.

The processor 56 determines whether the cuff 70 is pressurized at the target speed after the valve 16 is closed and the pump 14 is driven (Step ST12). For example, the processor 56 calculates an amount of change in the pressure in the cuff 70 detected by the pressure sensor 17 connected to the cuff 70 to a time and compares the amount of change in the pressure in the cuff 70 with the pressurization speed of the cuff 70 stored in the storage unit 54 in advance to determine whether or not the cuff 70 is pressurized at the target speed. When the cuff 70 is not pressurized at the target speed, for example, when the pressurization speed is faster or slower than the target speed (NO in Step ST12), the corrected frequency signal and voltage signal are output to the control circuit 60 (Step ST13).

The corrected frequency signal and voltage signal may be read by the processor 56 based on a data table stored in the storage unit 54 to output the frequency signal and the voltage signal for correction. Alternatively, the processor 56 may calculate the frequency signal and the voltage signal corrected according to a recording medium or the like or may generate the corrected frequency signal and voltage signal by another method.

The control circuit 60 outputs the corrected voltage signal to the booster circuit 59. When the boost voltage based on the voltage signal corrected by the booster circuit 59 is input, the control circuit 60 generates the rectangular signal based on the frequency signal and the boost voltage, and drives the pump 14 with the corrected rectangular signal (Step ST32).

When the cuff 70 is pressurized at the target speed (YES in Step ST12), the processor 56 determines whether or not the blood pressure measurement has ended (Step ST14). When the blood pressure measurement has not ended (NO in Step ST14), the processor 56 returns to Step ST12 and determines whether or not the cuff 70 is pressurized at the target speed.

When the blood pressure measurement has ended (YES in Step ST14), the processor 56 outputs the stop signal to the control circuit 60 (Step ST15). When the stop signal is input to the control circuit 60, the control circuit 60 outputs the stop signal to the booster circuit 59. When the stop signal is input, the booster circuit 59 has no boost voltage generated with the voltage signal, that is, does not generate the boost voltage. Therefore, the boost voltage is not input to the valve 16, and the valve 16 is opened (Step ST22). In addition, the control circuit 60 does not receive the boost voltage from the booster circuit 59 and stops generating the rectangular signal. As a result, the pump 14 stops (Step ST33), and the blood pressure measurement ends.

According to the blood pressure measurement device 1 including the drive block (drive circuit) 58 configured as described above, the boost voltage output from one booster circuit 59 can be used for driving the valve 16 and generating the rectangular signal for driving the pump 14. That is, the drive voltage (first drive signal) of the valve 16 and the drive signal (rectangular signal, second drive signal) of the pump 14 have the same voltage value. That is, a waveform of the drive voltage (first drive signal) of the valve 16 and an envelope of the drive signal (rectangular signal, second drive signal) of the pump 14 have a common shape changing at the same timing.

Specifically, as illustrated in FIG. 4, the waveform of the drive signal (first drive signal) for driving the valve 16 and the envelope of the peak voltage of the rectangular signal, which is the drive signal (second drive signal) for driving the pump 14, indicated by the dash-dotted line in FIG. 4 are inclined changing at the same timing.

Therefore, the booster circuit 59 and the control circuit 60 can be integrally configured in one circuit block. Therefore, the drive block 58, which is a drive circuit for driving the pump 14 and the valve 16, can be reduced in size. In addition, the number of components for configuring the drive block 58 that drives the pump 14 and the valve 16 can be reduced. Therefore, since the drive block 58 can be reduced in size, the blood pressure measurement device 1 can be reduced in size.

When blood pressure measurement is performed using the blood pressure measurement device 1, air is preferably supplied to the inside of the cuff 70 at a constant speed to pressurize the cuff. Therefore, the drive voltage of the pump 14 is gradually increased. The lowest voltage value of the drive voltage of the pump 14 is set higher than a drive voltage for switching the valve 16 from the open state to the closed state or a voltage value for maintaining the closed state after the valve 16 is driven to the closed state. In the present embodiment, after the input voltage is boosted to a voltage for driving the valve 16 from the open state to the closed state by the booster circuit 59 to drive the valve 16, the boost voltage is lowered to a voltage value higher than the lower limit voltage at which the closed state of the valve 16 can be maintained to generate the rectangular signal to drive the pump 14. Since the pump 14 can be driven by gradually increasing the voltage value of the boost voltage for driving the pump 14, it is possible to drive the pump 14 and maintain the closed state of the valve 16. In this way, the drive block 58 can generate the boost voltage for driving the pump 14 and the valve 16 by one booster circuit 59.

The blood pressure measurement device 1 uses the normally open type valve 16 for exhausting the air in the cuff 70. Therefore, the blood pressure measurement device 1 can stop supplying air from the pump 14 to the cuff 70 by stopping the drive block 58 at the time of abnormality, and can also open the valve 16 to rapidly exhaust the air in the cuff 70.

As described above, the drive block (drive circuit) 58 and the blood pressure measurement device 1 according to the present embodiment allow micrifying the circuit area reducing the number of components, and the pump 14 and the valve 16 can be driven by one drive block (drive circuit) 58.

### Other Embodiments

Note that the present invention is not limited to the embodiments described above. For example, in the example described above, the example in which the drive voltage for switching the valve 16 from the open state to the closed state is set to be higher than the voltage of the drive signal output to the pump 14 at the start of blood pressure measurement, and the drive voltage for switching the valve 16 from the closed state to the open state is set to 0 V or lower than the voltage of the drive signal output to the pump 14 during blood pressure measurement has been described, but the present invention is not limited thereto.

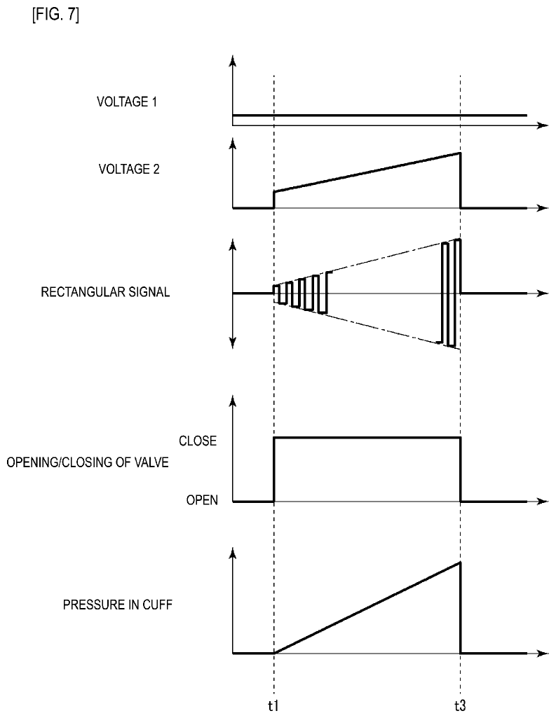

For example, as illustrated in an example of the control of the blood pressure measurement device 1 according to the second embodiment illustrated in FIG. 7, the drive voltage for switching the valve 16 from the open state to the closed state may be set to be the same as the voltage for driving the pump 14 at the blood pressure measurement start time t1 or lower than the voltage for driving the pump 14. When the valve 16 is used, as illustrated in FIG. 7, at the blood pressure measurement start time t1, the booster circuit 59 boosts the voltage to the voltage for driving the pump 14, and outputs the boosted voltage to the control circuit 60 and the valve 16, thereby driving the pump 14 and closing the valve 16. While the pump 14 is driven, the closing of the valve 16 is maintained.

The normally closed type valve 16 may be used. That is, as long as the pump 14 and the valve 16 are configured to be driven by the drive block 58 at the same voltage value, the type and the usage method of the valve 16 can be appropriately set. For example, when the valve 16 is a normally closed type, the valve 16 is disposed in the flow path between the pump 14 and the cuff 70.

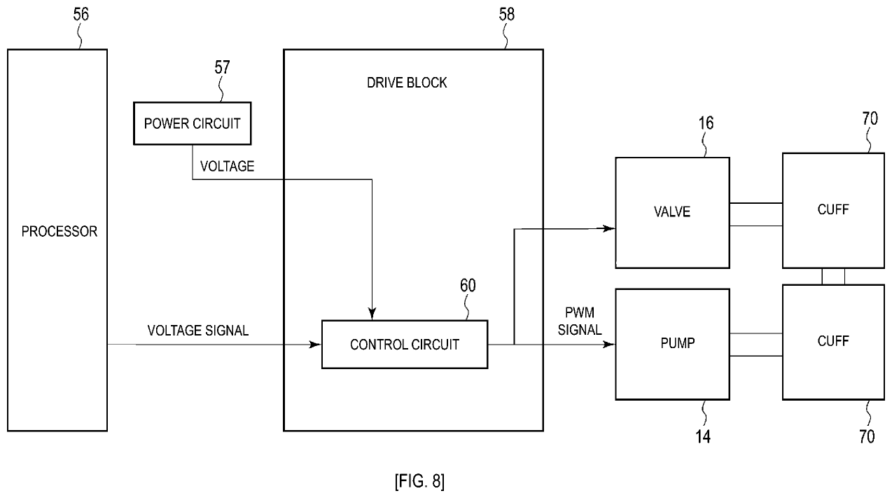

In addition, for example, although the example in which the pump 14 and the valve 16 are driven by the drive block 58 has been described in the above-described example, a plurality of the pumps 14 and a plurality of the valves 16 may be provided. That is, even when the blood pressure measurement device 1 is configured to include a plurality of one or both of the pumps 14 and the valves 16, the plurality of pumps 14 and/or the plurality of valves 16 can be driven by one drive block 58 as long as they are controlled to be driven at the same voltage value. The single cuff 70 or a plurality of the cuffs 70 may be connected to the pump 14 and the valve 16. When the plurality of cuffs 70 are connected to the pump 14 and the valve 16, for example, as in an example illustrated in FIG. 8, the plurality of cuffs 70 may be connected in series or the plurality of cuffs 70 may be connected in parallel (not illustrated).

In the above-described example, the example in which the transformer circuit 59 of the drive block (drive circuit) 58 is the booster circuit has been described, but the transformer circuit 59 is not limited thereto. For example, the transformer circuit 59 may be a step-down circuit or a step-up/down circuit. That is, as long as the voltage output from the power source circuit 57 can be transformed into the voltage value for driving the pump 14 and the valve 16, the transformer circuit 59 can be appropriately set.

Additionally, in the example described above, the configuration in which the drive block 58 includes the transformer circuit 59 has been described, but the drive block 58 is not limited thereto. For example, when the drive voltage of the valve 16 and the pump 14 is lower than the output voltage of the power source circuit 57, as illustrated in FIG. 8, the drive block 58 need not include a transformer circuit. In this case, for example, the control circuit 60 generates a Pulse Width Modulation (PWM) signal having an effective voltage corresponding to the voltage signal output from the processor 56. That is, the effective voltage corresponding to the voltage signal is obtained by controlling a duty ratio (equal to pulse width/circle) of the rectangular signal.

Then, the control circuit 60 outputs the PWM signal to the valve 16 and the pump 14 as a drive signal. The effective voltage is equal to or more than the voltage required for the valve 16 to operate, and generated as a voltage suitable for controlling an amount of air exhausted from the pump 14. For example, like the voltage 2 and the rectangular signal illustrated in FIG. 4 and FIG. 7, the voltage value of the effective voltage (effective value) may be gradually increased, or the effective voltage may be controlled to a voltage value (effective value) corresponding to the drive voltage of the valve 16 and after that controlled to a voltage value (effective value) corresponding to the drive voltage of the pump 14. For example, the rotary type pump 14 and the solenoid type valve 16 can be used. Thus, the drive block 58 including the control circuit 60 can drive the pump 14 and the valve 16 with one PWM signal. In addition, the drive block 58 configured as the control circuit 60 that controls the duty ratio eliminates the need for the transformer circuit 59, and therefore the size can be reduced and the number of components can be reduced.

That is, the present invention is not limited to the above-described embodiments, and various modifications can be made in an implementation stage without departing from the gist thereof. Furthermore, each of the embodiments may be carried out as appropriate in a combination as much as possible, and combined effects can be obtained in such a case. Furthermore, the inventions at various stages are included in the above-described embodiments, and the various inventions can be extracted in accordance with appropriate combinations in the plurality of disclosed constituent elements.

### REFERENCE NUMERALS LIST

1 Blood pressure measurement device
2 Device body
4 Fixture
5 Curler
6 Cuff structure
11 Case
12 Display device
13 Operation device
14 Pump
15 Flow path unit
16 Valve
17 Pressure sensor
18 Power supply unit
19 Communication device
20 Control board
31 Outer case
31a Lug
32 Windshield
41 Button
51 Substrate
54 Storage unit
55 Control unit
56 Processor
57 Power source circuit
58 Drive block (drive circuit)
59 Transformer circuit (booster circuit)
60 Control circuit
61 First belt
61a Belt portion
61b Buckle
61e Frame body
61f Prong
62 Second belt
62a Small hole
70 Cuff
71 Pressing cuff
72 Back plate
73 Sensing cuff
200 Wrist (living body)

1. A drive circuit for measuring blood pressure that generates a first drive signal for driving a valve for opening and closing a flow path connected to a blood pressure measurement cuff and a second drive signal for driving a pump for supplying a fluid to the cuff, wherein
the first drive signal and the second drive signal are generated from a common power supply voltage supplied from a power source circuit, and a waveform of the first drive signal and an envelope of a peak voltage of the second drive signal have a common shape changing at a same timing.

2. The drive circuit for measuring blood pressure according to claim 1, comprising:
a control circuit that outputs the second drive signal; and
a transformer circuit that transforms the power supply voltage into a voltage value corresponding to the first drive signal and the second drive signal and outputs the transformed voltage to the control circuit and the valve.

3. The drive circuit for measuring blood pressure according to claim 2, wherein
the transformer circuit is a booster circuit that boosts the power supply voltage.

4. The drive circuit for measuring blood pressure according to claim 2, wherein
the transformer circuit gradually increases the voltage value output to the valve and the control circuit.

5. The drive circuit for measuring blood pressure according to claim 3, wherein
the transformer circuit gradually increases the voltage value output to the valve and the control circuit.

6. The drive circuit for measuring blood pressure according to claim 2, wherein
a voltage value at which the valve is driven is higher than a voltage at a start of driving the pump; and
the transformer circuit transforms the power supply voltage into the voltage value at which the valve is driven and subsequently transforms the voltage value to a voltage value at which driving of the valve is maintained and the pump is driven.

7. The drive circuit for measuring blood pressure according to claim 3, wherein
a voltage value at which the valve is driven is higher than a voltage at a start of driving the pump; and
the transformer circuit transforms the power supply voltage into the voltage value at which the valve is driven and subsequently transforms the voltage value to a voltage value at which driving of the valve is maintained and the pump is driven.

8. The drive circuit for measuring blood pressure according to claim 4, wherein
a voltage value at which the valve is driven is higher than a voltage at a start of driving the pump; and
the transformer circuit transforms the power supply voltage into the voltage value at which the valve is driven and subsequently transforms the voltage value to a voltage value at which driving of the valve is maintained and the pump is driven.

9. The drive circuit for measuring blood pressure according to claim 5, wherein
a voltage value at which the valve is driven is higher than a voltage at a start of driving the pump; and
the transformer circuit transforms the power supply voltage into the voltage value at which the valve is driven and subsequently transforms the voltage value to a voltage value at which driving of the valve is maintained and the pump is driven.

10. The drive circuit for measuring blood pressure according to claim 2, wherein
the control circuit outputs a PWM signal to the pump and the valve as the first drive signal and the second drive signal, and the PWM signal has an effective voltage equal to or more than a voltage required for the valve and the pump to operate.

11. The drive circuit for measuring blood pressure according to claim 10, wherein
the control circuit gradually increases a voltage value of the effective voltage output to the valve and the pump.

12. The drive circuit for measuring blood pressure according to claim 10, wherein
a voltage value at which the valve is driven is higher than a voltage at a start of driving the pump; and
the control circuit sets the effective voltage to the voltage value at which the valve is driven and subsequently sets the voltage value to a voltage value at which driving of the valve is maintained and the pump is driven.

13. The drive circuit for measuring blood pressure according to claim 11, wherein
a voltage value at which the valve is driven is higher than a voltage at a start of driving the pump; and
the control circuit sets the effective voltage to the voltage value at which the valve is driven and subsequently sets the voltage value to a voltage value at which driving of the valve is maintained and the pump is driven.

14. A blood pressure measurement device, comprising:
a cuff to which a fluid is supplied;
a pump that supplies the fluid to the cuff;
a valve that opens and closes a flow path connected to the cuff;
a power source circuit;
the drive circuit for measuring blood pressure according to claim 1; and
a processor that outputs a control signal of a voltage to the drive circuit for measuring blood pressure.
